(19) United States
(12) Patent Application Publication
(10) Pub. No.: US 2015/0150768 A1
(43) Pub. Date: Jun. 4, 2015

(54) Furan Based Composition

(71) Applicants: Los Alamos National Security LLC; The Procter & Gamble Company, Cincinnati, OH (US)

(72) Inventors: Ryan M. West, West Chester, OH (US); Rulian Wu, Los Alamos, NM (US); Louis Alfred Silks, III, Los Alamos, NM (US)

(73) Assignees: Los Alamos National Security LLC; The Procter & Gamble Company, Cincinnati, OH (US)

(21) Appl. No.: 14/096,383

(22) Filed: Dec. 4, 2013

(51) Int. Cl. A61K 8/49; A61Q 15/00; A61Q 5/02; A61Q 19/10; A61Q 1/14; C11D 3/20; A61Q 5/12; A61Q 9/02

(52) U.S. Cl. CPC A61K 8/4973; A61Q 5/12; A61Q 15/00; A61Q 9/02; A61Q 19/10; A61Q 1/14; C11D 3/2096; A61Q 5/02

(57) ABSTRACT

Disclosed herein is a furan-based chemical comprising a furan group, hydrophilic group and hydrophobic group, wherein the hydrophilic group can be ionic, zwitterionic, or nonionic, and further, and wherein said hydrophobic group can be alkyl or alkenyl, linear or branched moeities

This invention was made under a CRADA (number LA01C10461-A001) between The Procter & Gamble Company and Los Alamos National Laboratories operated for the United States Department of Energy. The Government has certain rights in this invention.

### FIELD OF THE INVENTION

The disclosure generally relates to a novel furan-based composition. More specifically, the disclosure relates to a furan-based chemical comprising a furan group, hydrophilic group and hydrophobic group, wherein the hydrophilic group can be ionic, zwitterionic, or nonionic, and further, wherein said hydrophobic group can be alkyl or alkenyl, linear or branched moieties.

### BACKGROUND OF THE INVENTION

Linear alkyl benzene sulfonate (LAS) is an important surfactant for a variety of applications and represents around a quarter (579 thousand metric tons in 2009) of the surfactant used in household detergents, please see Chemical Economics Handbook, Surfactants, Household Detergents and their Raw Materials. The raw materials used to make LAS include alkenes, benzene and sulfur trioxide. Benzene is classified by the US department of Health and Human Services as a carcinogen in humans, therefore finding a suitable alternative to benzene is of strong interest

### SUMMARY OF THE INVENTION

One surprising alternative that can be used in place of benzene is furan. Furan groups contain an oxygen atom along with carbon atoms and are aromatic (i.e. a hetero atomic aromatic compound). This group serves to increase the hydrophilicity of the ring compared to benzene, and increase the electronic charge in the aromatic group. The aromatic group can be sulfonated to introduce a hydrophilic anionic group, while a hydrophobic group, such as an alkyl group, can also be introduced to create a molecule that acts as a surfactant. The substitution of furan for benzene represents a new class of surfactants with several benefits over the use of benzene. Benefits of this molecular scaffold include: 1) furan is more hydrophilic than benzene making the furan based structures more soluble in water; 2) furan based structures demonstrate good behavior as surfactants when compared to standard blends of benzene based structures, LAS; 3) furan can be derived from non petroleum sources such as furfural.

In one embodiment, the present invention is a furan-based chemical comprising a furan group, hydrophilic group and hydrophobic group, wherein the hydrophilic group can be ionic, zwitterionic, or nonionic, and further, wherein said hydrophobic group can be alkyl or alkenyl, linear or branched moieties and said hydrophobic group has between 6 and 26 carbons.

In another embodiment, the furan-based chemical is selected from the group consisting of structures depicted in Formulas I-VI, and mixtures thereof:

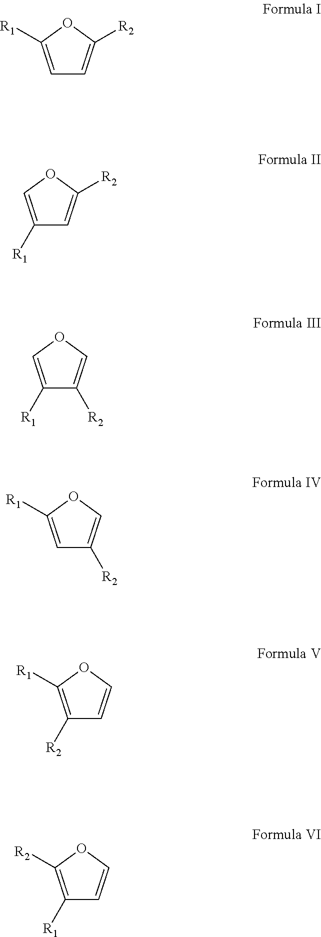

wherein:
R₁ is a hydrophilic group selected from the group consisting of —SO₃Na, —OSO₃Na, —CH₂OSO₃Na, —CH₂O(CH₂CH₂O)ₙH, —CH₂O(CH₂CH₂O)ₙSO₃Na and mixtures thereof wherein n is 1 to 6;
R₂ is a hydrophobic group with the structure of Formula VII wherein * denotes the site of attachment to Formula I-VI:

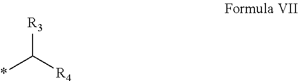

wherein:
R₃ is —(CH₂)ₘH wherein m is 0 to 9;
R₄ has the chemical structure of Formula VIII wherein W denotes the site of attachment to Formula VII:

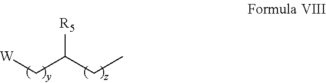

wherein:
R₅ is —(CH₂)ₚH wherein p is 0 to 9;
y is 0 to 16;
z is 0 to 16; and
wherein for R₂, the sum of m, p, y, and z is greater or equal to 3 and less than or equal to 23.

In one embodiment, the furan-based chemical has the structure of Formula I. In another embodiment, R₁ comprises —SO₃Na in the furan-based chemical, based on the chemical structures described above. In another embodiment, R₂ is an alkyl chain with 10 to 16 carbons in the furan-based chemical, based on the chemical structures described above.

In another embodiment, the furan-based chemical has the structure of Formula I and R₁ comprises —SO₃Na. In another embodiment, the furan-based chemical has the structure of Formula I and R₂ is an alkyl chain with 10 to 16 carbons.

In one embodiment, the present invention is a composition comprising

(a) from about 0.001 wt % to about 99.999 wt % of the furan-based chemical, and
(b) from about 0.001 wt % to about 99.999 wt % of at least one additional component selected from the group consisting of cleaning components and personal care components.

Useful cleaning components include surfactants, carriers, enzymes, builders, alkalinity systems, organic polymeric compounds, hueing dyes, bleaching compounds, alkanolamines, soil suspension agents, anti-redeposition agents, corrosion inhibitors, and mixtures thereof.

In one embodiment, the composition containing the furan-based chemical is selected from the group consisting of a granular detergent, a bar-form detergent, a liquid laundry detergent, a liquid hand dishwashing mixture, a hard surface cleaner, a tablet, a disinfectant, an industrial cleaner, a highly compact liquid, a powder, and a decontaminant.

In one embodiment, the composition containing the furan-based chemical may be used to launder soiled fabrics. In this embodiment, soiled fabrics are contacted with an effective amount of the composition in an aqueous solution.

In another embodiment, useful personal care components include oils, emollients, moisturizers, carriers, extracts, vitamins, minerals, anti-aging compounds, surfactants, solvents, polymers, preservatives, antimicrobials, waxes, particles, colorants, dyes, fragrances, and mixtures thereof.

In one embodiment, the composition containing the furan-based chemical is selected from the group consisting of a shampoo, a hair conditioner, a hair treatment, a facial soap, a body wash, a body soap, a foam bath, a make-up remover, a skin care product, an acne control product, a deodorant, an antiperspirant, a shaving aid, a cosmetic, a depilatory, a fragrance, a lotion, and mixtures thereof.

In one embodiment, the composition containing the furan-based chemical may be used to treat, cleanse and/or condition skin or hair. In this embodiment, skin or hair is contacted with an effective amount of the composition.

Additional features of the invention may become apparent to those skilled in the art from a review of the following detailed description, taken in conjunction with the examples and the appended claims.

### DETAILED DESCRIPTION OF THE INVENTION

The furan-based chemical comprising a furan group, hydrophilic group and hydrophobic group, wherein the hydrophilic group can be ionic, zwitterionic, or nonionic, and further, wherein said hydrophobic group can be alkyl or alkenyl, linear or branched moieties and said hydrophobic group has between 6 and 26 carbons.

Additional information on hydrophobic and hydrophilic groups can be found in the Kirk-Othmer Encyclopedia of Chemical Technology, specifically in the chapter on Surfactants, Household Detergents and their Raw Materials incorporated herein by reference.

Furan groups can be found in or produced from a variety of renewable biomass derived materials. The formation of a furan group from renewable biomass derived materials includes dehydration of a species containing at least four adjacent carbon atoms with a hydroxyl group on each of the four carbon atoms. A dehydration between the hydroxyl groups on the first and fourth carbons forms a carbon-oxygen-carbon group, while dehydration of the hydroxyl groups and adjacent hydrogen groups on the second and first, and third and four carbons, respectively, forms a double bond between the second and first and third and fourth carbons. Thus, species such as four, five and six carbon sugars, or four, five or six carbon polyols can be dehydrated to form furans. Specific examples include xylose dehydration to furfural and glucose or fructose dehydration to hydroxymethlyfurfural. Alternatively, furans can also be made directly from biomass, such as the well known Quaker Oats process to make furfural from agricultural residues, or pyrolysis process that make furanic compounds as components of pyrolysis oil.

### DEFINITIONS

As used herein, “hydrophobic” means a portion of a molecule that is generally insoluble in water and usually a hydrocarbon.

As used herein, “hydrocarbon” means a moiety that contains between 6 and 26 carbons, can be linear, branched or cyclic, and contains only carbon and hydrogen atoms.

As used herein, “alkyl” means each carbon in the hydrocarbon group is bound to four other atoms.

As used herein, “alkenyl” means at least two carbons in the hydrocarbon group are each bound by a single bond to two other atoms, and a double bond to each other.

As used herein, “hydrophilic” means a water soluble portion of a molecule that can either carry a formal charge, ionic, or can be neutral, non-ionic.

As used herein, “ionic” means a hydrophilic group that carries a formal positive charge, negative charge or both.

As used herein, “anionic” means a hydrophilic group that is typically a neutralized acid and has a negative charge that is balanced by a positive counterion. Anionic hydrophilic groups are the most commonly used type of hydrophilic group in surfactants. Typical anionic hydrophilic groups include but are not limited to the sodium (Na⁺) form of carboxylic acids, sulfates, sulfonates, and phosphates.

As used herein, “cationic” means a hydrophilic group that has a positive charge and is balanced by a negative counter ion, for example chloride (Cl⁻). Typical cationic hydrophilic groups are quaternary ammonium compounds that contain a nitrogen group bound to 4 other atoms.

As used herein, “zwitterionic” means a hydrophilic groups that contains both cationic and anionic groups.

As used herein, “nonionic” means a hydrophilic group that does not contain a formal charge like the ionic groups. Typically, nonionic groups contain carbon, hydrogen, oxygen and nitrogen, with the most common form being based on ethylene oxide to form ethoxylates. The ethoxylate hydrophilic group is typically connected via an ether linkage to the rest of the molecule, but can also be connected via an ester, amine, or amide linkage. Other nonionic groups can be amine ethoxylates, polyols and polyol derivatives, such as glycerol, propanediol, xylitol, sorbitol, mono and poly-saccharide derivates, such as glucose, sucrose, maltose, or xylose derivatives, and polyol amines, such as glucamine or xylosamine.

In one embodiment, the present invention is a furan-based chemical comprising a furan group, hydrophilic group and hydrophobic group, wherein the hydrophilic group can be ionic, zwitterionic, or nonionic, and further, wherein said hydrophobic group can be alkyl or alkenyl, linear or branched moieties and said hydrophobic group has between 6 and 26 carbons.

In another embodiment, the furan-based chemical is selected from the group consisting of structures depicted in Formulas I-VI, and mixtures thereof:

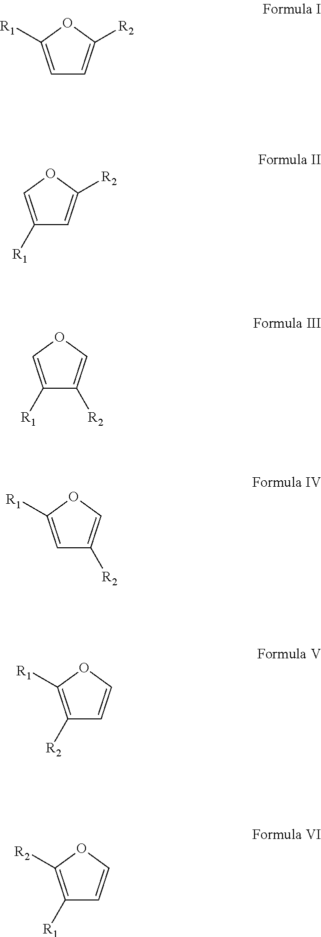

wherein:
R₁ is a hydrophilic group selected from the group consisting of —SO₃Na, —OSO₃Na, —CH₂OSO₃Na, —CH₂O(CH₂CH₂O)ₙH, —CH₂O(CH₂CH₂O)ₙSO₃Na and mixtures thereof wherein n is 1 to 6;
R₂ is a hydrophobic group with the structure of Formula VII wherein * denotes the site of attachment to Formula I-VI:

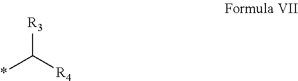

wherein:
R₃ is —(CH₂)ₘH wherein m is 0 to 9;
R₄ has the chemical structure of Formula VIII wherein W denotes the site of attachment to Formula VII:

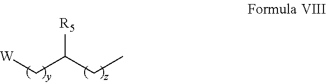

wherein:
R₅ is —(CH₂)ₚH wherein p is 0 to 9;
y is 0 to 16;
z is 0 to 16; and
wherein for R₂, the sum of m, p, y, and z is greater or equal to 3 and less than or equal to 23.

In one embodiment, the furan-based chemical has the structure of Formula I. In another embodiment, R₁ comprises —SO₃Na in the furan-based chemical, based on the chemical structures described above. In another embodiment, R₂ is an alkyl chain with 10 to 16 carbons in the furan-based chemical, based on the chemical structures described above.

In another embodiment, the furan-based chemical has the structure of Formula I and R₁ comprises —SO₃Na. In another embodiment, the furan-based chemical has the structure of Formula I and R₂ is an alkyl chain with 10 to 16 carbons.

In one embodiment, the present invention is a composition comprising

(a) from about 0.001 wt % to about 99.999 wt % of the furan-based chemical, and
(b) from about 0.001 wt % to about 99.999 wt % of at least one additional component selected from the group consisting of cleaning components and personal care components.

Useful cleaning components include surfactants, carriers, enzymes, builders, alkalinity systems, organic polymeric compounds, hueing dyes, bleaching compounds, alkanolamines, soil suspension agents, anti-redeposition agents, corrosion inhibitors, and mixtures thereof.

In one embodiment, the composition containing the furan-based chemical is selected from the group consisting of a granular detergent, a bar-form detergent, a liquid laundry detergent, a liquid hand dishwashing mixture, a hard surface cleaner, a tablet, a disinfectant, an industrial cleaner, a highly compact liquid, a powder, and a decontaminant.

In one embodiment, the composition containing the furan-based chemical may be used to launder soiled fabrics. In this embodiment, soiled fabrics are contacted with an effective amount of the composition in an aqueous solution.

In another embodiment, useful personal care components include oils, emollients, moisturizers, carriers, extracts, vitamins, minerals, anti-aging compounds, surfactants, solvents, polymers, preservatives, antimicrobials, waxes, particles, colorants, dyes, fragrances, and mixtures thereof.

In one embodiment, the composition containing the furan-based chemical is selected from the group consisting of a shampoo, a hair conditioner, a hair treatment, a facial soap, a body wash, a body soap, a foam bath, a make-up remover, a skin care product, an acne control product, a deodorant, an antiperspirant, a shaving aid, a cosmetic, a depilatory, a fragrance, a lotion, and a mixtures thereof.

In one embodiment, the composition containing the furan-based chemical may be used to treat, cleanse and/or condition skin or hair. In this embodiment, skin or hair is contacted with an effective amount of the composition.

### Surfactant Compositions and Products

Furan-based surfactant compositions comprising one or more derivatives of a detergent alcohol selected from the anionic, nonionic, cationic, amine oxide, and or zwitterionic mixtures thereof are outstandingly suitable as soil detachment and suspending additives for laundry and other cleaning compositions. The dialkyl or diester quats are particularly well suited for fabric softener compositions.

The furan-based surfactant compositions according to the present invention can be added to the laundry detergents, cleaning compositions, and fabric softener compositions in amounts of generally from 0.05 to 70% by weight, preferably from 0.1 to 40% by weight and more preferably from 0.25 to 10% by weight, based on the particular overall composition.

In addition, the laundry detergents and cleaning compositions generally comprise surfactants and, if appropriate, other polymers as washing substances, builders and further customary ingredients, for example cobuilders, cleaning polymers (modified and unmodified polycarboxylates, ethoxylated amines and derivatives thereof), complexing agents, bleaches, standardizers, graying inhibitors, dye transfer inhibitors, enzymes and perfumes.

The novel surfactant compositions of the present invention may be utilized in laundry detergents or cleaning compositions comprising a surfactant system comprising alkyl furan sulfonates (AFS) and one or more co-surfactants selected from nonionic, cationic, anionic or mixtures thereof. The selection of co-surfactant may be dependent upon the desired benefit. In one embodiment, the co-surfactant is selected as a nonionic surfactant, preferably C12-C18 alkyl ethoxylates. In another embodiment, the co-surfactant is selected as an anionic surfactant, preferably C10-C18 alkyl alkoxy sulfates (AExS) wherein x is from 1-30. In another embodiment the co-surfactant is selected as a cationic surfactant, preferably dimethyl hydroxyethyl lauryl ammonium chloride. If the surfactant system contains AFS, the AFS is used at levels ranging from about 1% to about 25%, or from about 3% to about 25%, or from about 5% to about 23% by weight of the composition.

The surfactant system may comprise from 0% to about 7%, or from about 0.1% to about 5%, or from about 1% to about 4% by weight of the composition of a co-surfactant selected from a nonionic co-surfactant, cationic co-surfactant, anionic co-surfactant and any mixture thereof.

Non-limiting examples of nonionic co-surfactants include: C12-C18 alkyl ethoxylates, such as, NEODOL® nonionic surfactants from Shell; C6-C12 alkyl phenol alkoxylates wherein the alkoxylate units are a mixture of ethyleneoxy and propyleneoxy units; C12-C18 alcohol and C6-C12 alkyl phenol condensates with ethylene oxide/propylene oxide block alkyl polyamine ethoxylates such as PLURONIC® from BASF; C14-C22 mid-chain branched alcohols, BA, as discussed in U.S. Pat. No. 6,150,322; C14-C22 mid-chain branched alkyl alkoxylates, BAEx, wherein x is from 1-30, as discussed in U.S. Pat. No. 6,153,577, U.S. Pat. No. 6,020,303 and U.S. Pat. No. 6,093,856; alkylpolysaccharides as discussed in U.S. Pat. No. 4,565,647 Llenado, issued Jan. 26, 1986; specifically alkylpolyglycosides as discussed in U.S. Pat. No. 4,483,780 and U.S. Pat. No. 4,483,779; polyhydroxy detergent acid amides as discussed in U.S. Pat. No. 5,332,528; and ether capped poly(oxyalkylated) alcohol surfactants as discussed in U.S. Pat. No. 6,482,994 and WO 01/42408.

Non-limiting examples of semi-polar nonionic co-surfactants include: water-soluble amine oxides containing one alkyl moiety of from about 10 to about 18 carbon atoms and 2 moieties selected from the group consisting of alkyl moieties and hydroxyalkyl moieties containing from about 1 to about 3 carbon atoms; water-soluble phosphine oxides containing one alkyl moiety of from about 10 to about 18 carbon atoms and 2 moieties selected from the group consisting of alkyl moieties and hydroxyalkyl moieties containing from about 1 to about 3 carbon atoms; and water-soluble sulfoxides containing one alkyl moiety of from about 10 to about 18 carbon atoms and a moiety selected from the group consisting of alkyl moieties and hydroxyalkyl moieties of from about 1 to about 3 carbon atoms (See WO 01/32816, U.S. Pat. No. 4,681,704, and U.S. Pat. No. 4,133,779).

Non-limiting examples of cationic co-surfactants include: the quaternary ammonium surfactants, which can have up to 26 carbon atoms include: alkoxylate quaternary ammonium (AQA) surfactants as discussed in U.S. Pat. No. 6,136,769; dimethyl hydroxyethyl quaternary ammonium as discussed in U.S. Pat. No. 6,004,922; dimethyl hydroxyethyl lauryl ammonium chloride; polyamine cationic surfactants as discussed in WO 98/35002, WO 98/35003, WO 98/35004, WO 98/35005, and WO 98/35006; cationic ester surfactants as discussed in U.S. Pat. Nos. 4,228,042, 4,239,660 4,260,529 and U.S. Pat. No. 6,022,844; and amino surfactants as discussed in U.S. Pat. No. 6,221,825 and WO 00/47708, specifically amido propyldimethyl amine (APA).

Non-limiting examples of anionic co-surfactants useful herein include: C10-C20 primary, branched chain and random alkyl sulfates (AS); C10-C18 secondary (2,3) alkyl sulfates; C10-C18 alkyl alkoxy sulfates (AExS) wherein x is from 1-30; C10-C18 alkyl alkoxy carboxylates comprising 1-5 ethoxy units; mid-chain branched alkyl sulfates as discussed in U.S. Pat. No. 6,020,303 and U.S. Pat. No. 6,060,443; mid-chain branched alkyl alkoxy sulfates as discussed in U.S. Pat. No. 6,008,181 and U.S. Pat. No. 6,020,303; modified alkylbenzene sulfonate (MLAS) as discussed in WO 99/05243, WO 99/05242 and WO 99/05244; methyl ester sulfonate (MES); and alpha-olefin sulfonate (AOS).

In another embodiment, the cleaning composition of the present invention is a liquid or solid laundry detergent composition. In another embodiment, the cleaning composition of the present invention is a hard surface cleaning composition, preferably wherein the hard surface cleaning composition impregnates a nonwoven substrate. As used herein “impregnate” means that the hard surface cleaning composition is placed in contact with a nonwoven substrate such that at least a portion of the nonwoven substrate is penetrated by the hard surface cleaning composition, preferably the hard surface cleaning composition saturates the nonwoven substrate. The cleaning composition may also be utilized in car care compositions, for cleaning various surfaces such as hard wood, tile, ceramic, plastic, leather, metal, glass. This cleaning composition could be also designed to be used in a personal care and pet care compositions such as shampoo composition, body wash, liquid or solid soap and other cleaning composition in which surfactant comes into contact with free hardness and in all compositions that require hardness tolerant surfactant system, such as oil drilling compositions.

In another embodiment the cleaning composition is a dish cleaning composition, such as liquid hand dishwashing compositions, solid automatic dishwashing compositions, liquid automatic dishwashing compositions, and tab/unit dose forms of automatic dishwashing compositions.

Quite typically, cleaning compositions herein such as laundry detergents, laundry detergent additives, hard surface cleaners, synthetic and soap-based laundry bars, fabric softeners and fabric treatment liquids, solids and treatment articles of all kinds will require several adjuncts, though certain simply formulated products, such as bleach additives, may require only, for example, an oxygen bleaching agent and a surfactant as described herein. A comprehensive list of suitable laundry or cleaning adjunct materials can be found in WO 99/05242.

Common cleaning adjuncts include builders, enzymes, polymers not discussed above, bleaches, bleach activators, catalytic materials and the like excluding any materials already defined hereinabove. Other cleaning adjuncts herein can include suds boosters, suds suppressors (antifoams) and the like, diverse active ingredients or specialized materials such as dispersant polymers (e.g., from BASF Corp. or Rohm & Haas) other than those described above, color speckles, silvercare, anti-tarnish and/or anti-corrosion agents, dyes, fillers, germicides, alkalinity sources, hydrotropes, anti-oxidants, enzyme stabilizing agents, pro-perfumes, perfumes, solubilizing agents, carriers, processing aids, pigments, and, for liquid formulations, solvents, chelating agents, dye transfer inhibiting agents, dispersants, brighteners, suds suppressors, dyes, structure elasticizing agents, fabric softeners, anti-abrasion agents, hydrotropes, processing aids, and other fabric care agents, surface and skin care agents. Suitable examples of such other cleaning adjuncts and levels of use are found in U.S. Pat. Nos. 5,576,282, 6,306,812 B1 and 6,326,348 B1.

### Method of Use

The present invention includes a method for cleaning a targeted surface. As used herein “targeted surface” may include such surfaces such as fabric, dishes, glasses, and other cooking surfaces, hard surfaces, hair or skin. As used herein “hard surface” includes hard surfaces being found in a typical home such as hard wood, tile, ceramic, plastic, leather, metal, glass. Such method includes the steps of contacting the composition comprising the modified polyol compound, in neat form or diluted in wash liquor, with at least a portion of a targeted surface then optionally rinsing the targeted surface. Preferably the targeted surface is subjected to a washing step prior to the aforementioned optional rinsing step. For purposes of the present invention, washing includes, but is not limited to, scrubbing, wiping and mechanical agitation.

As will be appreciated by one skilled in the art, the cleaning compositions of the present invention are ideally suited for use in home care (hard surface cleaning compositions) and/or laundry applications.

The composition solution pH is chosen to be the most complimentary to a target surface to be cleaned spanning broad range of pH, from about 5 to about 11. For personal care such as skin and hair cleaning pH of such composition preferably has a pH from about 5 to about 8 for laundry cleaning compositions pH of from about 8 to about 10. The compositions are preferably employed at concentrations of from about 150 ppm to about 10,000 ppm in solution. The water temperatures preferably range from about 5° C. to about 100° C.

For use in laundry cleaning compositions, the compositions are preferably employed at concentrations from about 150 ppm to about 10000 ppm in solution (or wash liquor). The water temperatures preferably range from about 5° C. to about 60° C. The water to fabric ratio is preferably from about 1:1 to about 20:1.

The method may include the step of contacting a nonwoven substrate impregnated with an embodiment of the composition of the present invention As used herein “nonwoven substrate” can comprise any conventionally fashioned nonwoven sheet or web having suitable basis weight, caliper (thickness), absorbency and strength characteristics. Examples of suitable commercially available nonwoven substrates include those marketed under the tradename SONTARA® by DuPont and POLYWEB® by James River Corp.

As will be appreciated by one skilled in the art, the cleaning compositions of the present invention are ideally suited for use in liquid dish cleaning compositions. The method for using a liquid dish composition of the present invention comprises the steps of contacting soiled dishes with an effective amount, typically from about 0.5 ml. to about 20 ml. (per 25 dishes being treated) of the liquid dish cleaning composition of the present invention diluted in water.

### EXAMPLES

The following examples are provided to illustrate the invention, but are not intended to limit the scope thereof. Various furan-based compositions were prepared as follows:

### Preparation of 2-(furan-2-yl)dodecanol

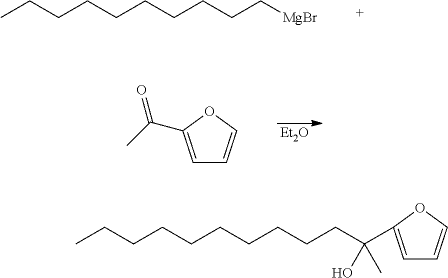

To decylmagnesium bromide solution (300 mL 1M in Et₂O, 0.3 moles, Aldrich), blanketed with nitrogen and chilled to <10° C. using ice/salt bath, was added 2-acetylfuran (33 g, 0.3 moles, Aldrich, 1:1 in Et₂O) at a rate so T<10° C. Once addition was complete, removed cooling bath and allowed solution to warm to room temperature (RT). Stirred at RT 1 hr. This was added to a solution of ammonium chloride (32.1 g, 0.6 moles, EMD, in 200 mL H₂O) and stirred at RT 1 hr. Resultant was transferred to separatory funnel and allowed to stand overnight. Aqueous layer was separated and organic layer was washed 2×100 mL 10% brine. The organic layer was dried over anhydrous magnesium sulfate, filtered and the solvent was removed. 75.5 g of tan oil resulted (99% yield, 89% product by GC, GC/MS).

### Preparation of a mixture of (E)-2-(dodec-2-en-2-yl)furan, (Z)-2-(dodec-2-en-2-yl)furan and 2-(dodec-1-en-2yl)furan

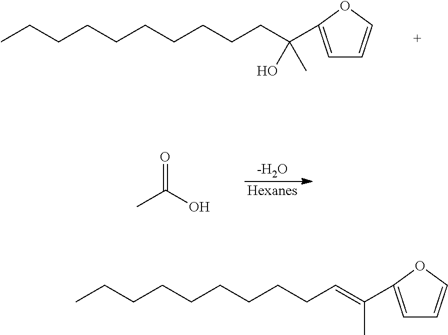

A solution of 2-(furan-2-yl)dodecanol (75.5 g, 89% pure, 0.11 moles), acetic acid (15 g, 20 wt % based on starting alcohol, Baker) in hexanes (200 ml) was refluxed 24 hrs, under nitrogen, during which time water was removed by azeotropic distillation with hexanes and collected in Dean-Stark trap. The resultant solution was cautiously added to saturated aqueous sodium bicarbonate solution (300 ml) and stirred 30 min at RT. The resultant solution was transferred to separatory funnel. The aqueous layer was separated and organic layer was washed 2×100 mL 10% brine. The organic layer was dried over anhydrous magnesium sulfate, filtered and stripped of solvent. 70.5 g brown oil resulted (98% yield, shown to be 87.9% mixture of (E)-2-(dodec-2-en-2-yl)furan, (Z)-2-(dodec-2-en-2-yl)furan and 2-(dodec-1-en-2yl)furan by GC GC/MS).

### Preparation of 2-(dodecan-2-yl)furan

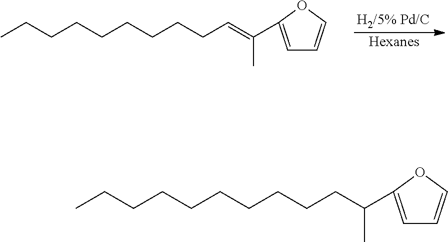

Charged 600 mL Parr reactor with a mixture of (E)-2-(dodec-2-en-2-yl)furan, (Z)-2-(dodec-2-en-2-yl)furan and 2-(dodec-1-en-2yl)furan (70 g, 0.3 moles), 300 mg 5% Pd/C (Aldrich) and 400 mL Hexanes. Purged reactor 3×100 PSI N₂ and 3×50 PSI H₂. Stirring was continued for 3 hrs, at RT, maintaining H₂ pressure between 10-50 PSI. Upon completion of reaction, the catalyst was removed by filtration. The solvent was removed giving rise to 63.8 g brown oil (72% product by GC GC/MS). The material was purified by distillation through a 6″ vigreux column yielding 45.6 g product oil at 124-126° C. and 2 mm Hg (92.9% pure by GC GC/MS). This was further purified by plug column chromatography (500 mL EMD's Silica gel 60, 230-400 mesh, Hexanes mobile phase, Rf=0.8) yielding 37.9 g colorless oil (53.9% yield, 98.6% pure by GC GC/MS).

### Preparation of sodium 5-(dodecan-2-yl)furan-2-sulfonate (C12 2-furan AFS)

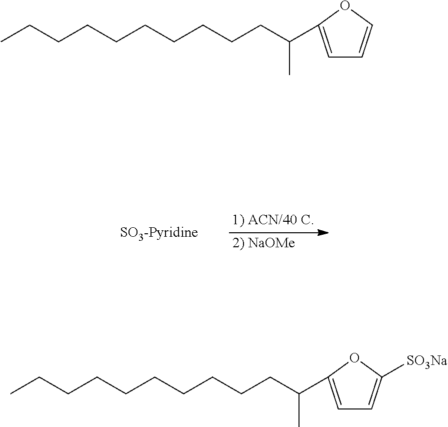

A slurry of 2-(dodecan-2-yl)furan (20 g, 0.084 moles), SO₃-pyridine complex (20 g, 0.126 moles) and anhydrous acetonitrile (25 mL) was stirred at 40° C. 24 hrs under nitrogen after which the slurry was filtered hot to remove insoluble material. The filtrate was cooled and added to a solution of NaOMe/MeOH (18.3 g 25% NaOMe/MeOH, Aldrich, 0.084 moles, 75 mL MeOH) and stirred at RT 30 min Concentrated to ˜70 ml. Filtered to remove insoluble salts. Added filtrate to 200 ml fresh acetonitrile. After standing at RT 1 hr a precipitant was filtered out and dried to 10.2 g (35.7% yield, H-NMR consistent w/product, 92.4% active by CAT SO₃ titration).

### Preparation of sodium 5-(undecan-2-yl)furan-2-sulfonate, (C11 2-furan AFS)

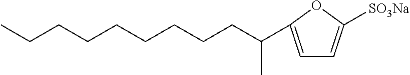

Used same process for the preparation of 5-(dodecan-2-yl)furan-2-sulfonate with exception of using nonyl-Grignard solution in initial reaction. Final product's H-NMR was consistent w/product and 87.8% active by CAT SO₃ titration.

### Purification of 2-dodecylfuran

10 g 2-dodecylfuran was purchased from 3-B Scientific Corp. This was shown to be 7.13% 1-bromododecane, 83.9% 2-dodecylfuran, 8.9% 2,5-di-(dodecyl)furan by GC GC/MS and was purified by distillation of thru 6″ vigreux column 4.0 g product oil was collected at 105-112° C. and 0.5 mm Hg (99.4% pure by GC GC/MS).

### Preparation of sodium 5-dodecanfuran-2-sulfonate (C12 1-furan AFS)

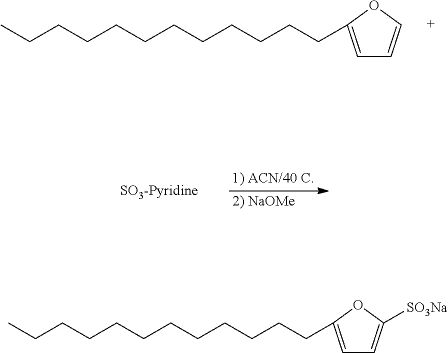

A slurry of 2-dodecylfuran (4 g, 0.017 moles), SO3-Pyridine complex (4 g, 0.026 moles) and anhydrous acetonitrile (10 mL) was stirred at 40° C. 24 hrs under nitrogen. The slurry was added hot to a solution of NaOMe/MeOH (5.6 g, 25% NaOMe/MeOH, Aldrich, 0.026 moles, 20 mL MeOH) and stirred at RT 30 min Solvent was removed by evaporation overnight. The resultant paste was dissolved in water (35 ml) at 70° C. After chilling resultant at 10° C. 2 hrs a white precipitant was collected by filtration and dried to 4.3 g. (75% yield, H-NMR consistent w/product, 97.6% active by CAT SO₃ titration).

### Preparation of sodium 5-tridecanfuran-2-sulfonate (C13-1-AFS)

Sodium 5-tridecanefuran-2-sulfonate was synthesized using the same process as sodium 5-dodecanfuran-2-sulfonate.

### Comparison to Other Surfactants

To demonstrate the superiority of the present invention vs previously disclosed or already on the market surfactants, solubility tests and dIFT (dynamic oil-water InterFacial Tension) measurements were performed. Methods are as shown below.

Solubility of System at 1 wt % with Hardness

Stock solutions of all species were made at 1% by weight in deionized water. Several species did not dissolve at room temperature. When heated to approximately 55° C. these species temporarily went into solution long enough to draw an aliquot of liquid. The 2000 μL aliquots were added to a 100 mL flask and diluted to 200 ppm using 6.122 grains per gallon (gpg) water. At this point, several of the solutions became cloudy and formed a precipitant within 10 minutes, making dIFT measurements impractical. These observations are summarized in Table 1. All the furan containing species, dubbed alkyl furan sulfonate (AFS) remained soluble after heating and addition of hardness for the duration of testing. Pure LAS species, C11 1-phenyl LAS, C12 2-phenyl LAS and C13 2-phenyl LAS did not remain soluble after addition of hardness. For this reason a current commercial LAS with an average of 11.8 carbons in the chain was chosen for comparison purposes. Additionally a modified LAS (MLAS) was chosen for comparison. MLAS structures have increased solublility and hardness tolerance as described by U.S. Pat. No. 6,306,817B1.

TABLE 1
1% Solution by weight in water

| | | | Soluble after |
|---|---|---|---|
| | | | addit on of |
| | | Soluble after | 6 gpg hardness |
| | Soluble at Room | heating to | (for at least |
| | Temperature | ≈55° C. | 30 minutes) |
| C17 1-furan A = S | No | Yes | Yes |
| C13 1-furan A = S | No | Yes | Yes |
| C11 2-furan A = S | Yes | Yes | Yes |
| C12 2-furan A = S | Yes | Yes | Yes |
| C11 1-phenyl LAS | No | Yes | No |
| C12 2-phenyl LAS | No | Yes | No |
| C13 2-phenyl LAS | No | Yes | No |
| C11.8 LAS | Yes | Yes | Yes |
| C12 1-phenyl MLAS | Yes | Yes | Yes |

Species that remained soluble in solution at 6 gpg of hardness also remained soluble in solution at 20 gpg of hardness for the duration of testing.

Method—Dynamic Interfacial Tension Analysis (dIFT)

Dynamic Interfacial Tension analysis was performed on a Krüss® DVT30 Drop Volume Tensiometer (Krüss USA, Charlotte, N.C.). The instrument was configured to measure the interfacial tension of an ascending oil drop in the aqueous surfactant solution. The oil used was canola oil (Crisco Pure Canola Oil manufactured by The J.M. Smucker Company). The aqueous surfactant and oil phases were temperature controlled at 21.1° C. (+/−1° C.), via a recirculating water temperature controller attached to the tensiometer. A dynamic interfacial tension curve was generated by dispensing the oil drops into the aqueous surfactant phase from an ascending capillary with an internal diameter of 0.2540 mm, over a range of flow rates and measuring the interfacial tension at each flow rate. Data is generated at oil dispensing flow rates of 500 μL/min to 1 μL/min with 2 flow rates per decade on a logarithmic scale (7 flow rates measured in this instance). Interfacial tension was measured on three oil drops per flow rate and then averaged. Interfacial tension was reported in units of mN/m. Surface age of the oil drops at each flow rate is also recorded and plots can be generated either of interfacial tension (y-axis) versus oil flow rate (x-axis) or interfacial tension (y-axis) versus oil drop surface age (x-axis). Minimum interfacial tension (mN/m) is the lowest interfacial tension at the slowest flow rate, with lower numbers indicating superior performance. Example of analysis of a 200 ppm surfactant concentration, with water hardness (3:1 Ca:Mg) of 6 or 20 grains per gallon (gpg) as noted, 21.1° C., pH 8.3-8.5: Sample was analyzed as described above. Density settings for 21.1° C. were set at 0.917 g/ml for Canola Oil and 0.998 g/ml for aqueous surfactant phase (assumed to be the same as water since dilute solution). To a 100 ml volumetric flask was added 2.00 mL of 1% (wt/wt) Surfactant solution in deionized water and the volumetric is then filled to the mark with 6.12 gpg water for target 6 gpg solution or 20.408 gpg water for target 20 gpg solution, (3:1 CaCl₂:MgCl₂ solution) and mixed well. The solution was transferred to a beaker and the pH adjusted to 8.3-8.5 by addition of a few drops of 0.1 N NaOH or 0.1 N H₂SO₄. The solution is then loaded into the tensiometer measurement cell and analyzed. Total time from addition of hardness to surfactant to time loaded into tensiometer is 5 minutes.

The following surfactants were analyzed via dIFT measurements. Surfactants were analyzed at a level of 200 ppm. Analysis conditions are in water of both 6 and 20 gpg Calcium/Magnesium water hardness level (3:1 calcium:magnesium), 21.1° C. and adjusted to pH 8.3-8.5.

### Materials:

1 wt % solutions of each of the following:

1. C12 2-furan AFS

2. C11 2-furan AFS

3. C12 1-furan AFS

4. C13 1-furan AFS

5. C11.8 LAS

6. C12 1-phenyl MLAS

TABLE 2
dIFT Measurements (mN/m) at varying Hardness

| | | IFT @ 1 μL/min |
|---|---|---|
| | Solution Hardness | Oil Flowrate, |
| Surfactant | (gpg, 3:1 Calcium:Magnesium) | 21.1° C. |
| C12 2-furan alkyl | 6 | 1.15 |
| furan sulfonate | 20 | 0.94 |
| C11 2-furan alkyl | 6 | 7.25 |
| furan sulfonate | 20 | 4.03 |
| C12 1-furan alkyl | 6 | 0.31 |
| furan sulfonate | 20 | 0.25 |
| C13 1-furan alkyl | 6 | 0.08 |
| furan sulfonate | 20 | 0.08 |
| C11.8 LAS | 6 | 1.39 |
| | 20 | 1.46 |
| C12 1-phenyl | 6 | 0.32 |
| MLAS | 20 | 0.89 |

For all AFS materials, there is high hardness tolerance up to 20 gpg evidenced by the decrease in min IFT from 6 to 20 gpg. Increasing the chain length of the AFS materials decreases min IFT up to C13. C12 and C13 alkyl furan sulfonates have minimum interfacial tensions that surpass those of the C11.8 LAS blend used, especially at 20 gpg. 1-furan AFS outperforms 2-furan AFS in terms of physical properties for grease cleaning.

Furan based surfactants represent several distinct advantages over benzene derived surfactants. First, as demonstrated in solubility tests, furan based structures are more soluble in water than the equivalent benzene derived structures. Second furan based structures demonstrate lower and hence better values in min IFT than commercial LAS blends that have a benzene based structures. Third, furans can be derived from non petroleum sources such as from sugars, polylos, or furfurals, where benzene is produced from petroleum

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention

What is claimed is:

1. A furan-based chemical comprising a furan group, hydrophilic group and hydrophobic group, wherein the hydrophilic group can be ionic, zwitterionic, or nonionic, and further, wherein said hydrophobic group can be alkyl or alkenyl, linear or branched moeities and said hydrophobic group has between 6 and 26 carbons.

2. The furan-based chemical of claim 1 wherein said chemical is selected from the group consisting of structures depicted in Formulas I-VI, and mixtures thereof: 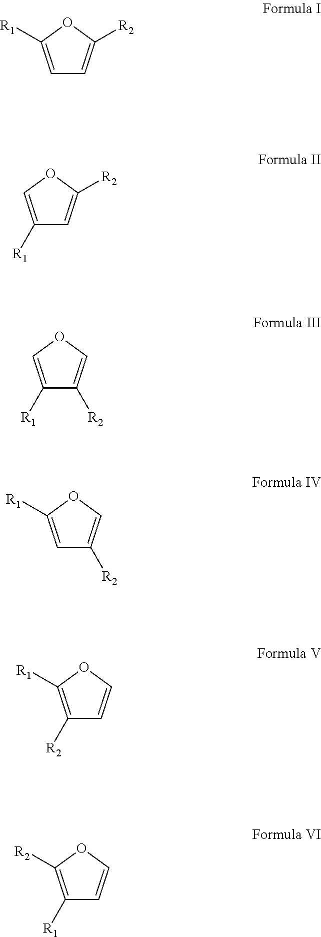
wherein:
R₁ is a hydrophilic group selected from the group consisting of —SO₃Na, —OSO₃Na, —CH₂OSO₃Na, —CH₂O(CH₂CH₂O)ₙH, —CH₂O(CH₂CH₂O)ₙSO₃Na and mixtures thereof wherein n is 1 to 6;
R₂ is a hydrophobic group with the structure of Formula VII wherein * denotes the site of attachment to Formula I-VI: 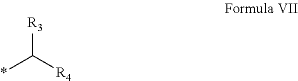
wherein:
R₃ is —(CH₂)ₚH wherein m is 0 to 9;
R₄ has the chemical structure of Formula VIII wherein W denotes the site of attachment to Formula VII: 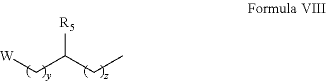
wherein:
R₅ is —(CH₂)ₚH wherein p is 0 to 9;
y is 0 to 16;
z is 0 to 16; and
wherein for R₂, the sum of m, p, y, and z is greater or equal to 3 and less than or equal to 23.

3. The furan-based chemical of claim 2, wherein said furan-based chemical has the structure of Formula I.

4. The furan-based chemical of claim 2, wherein R₁ comprises —SO₃Na.

5. The furan-based chemical of claim 2, wherein R₂ is an alkyl chain with 10 to 16 carbons.

6. The furan-based chemical of claim 3, wherein R₁ comprises —SO₃Na.

7. The furan-based chemical of claim 3, wherein R₂ is an alkyl chain with 10 to 16 carbons.

8. A composition comprising
(a) from about 0.001 wt % to about 99.999 wt % of the furan-based chemical of claim 1, and
(b) from about 0.001 wt % to about 99.999 wt % of at least one additional component selected from the group consisting of cleaning components and personal care components.

9. A composition comprising
(a) from about 0.001 wt % to about 99.999 wt % of the mixture of claim 2, and
(b) from about 0.001 wt % to about 99.999 wt % of at least one additional component selected from the group consisting of cleaning components and personal care components.

10. The composition of claim 8 or 9, wherein the at least one cleaning component is selected from the group consisting of a surfactant, a carrier, an enzyme, a builder, an alkalinity system, an organic polymeric compound, a hueing dye, a bleaching compound, an alkanolamine, a soil suspension agent, an anti-redeposition agent, a corrosion inhibitor, and mixtures thereof.

11. The composition of claim 8 or 9, wherein the composition is selected from the group consisting of a granular detergent, a bar-form detergent, a liquid laundry detergent, a liquid hand dishwashing mixture, a hard surface cleaner, a tablet, a disinfectant, an industrial cleaner, a highly compact liquid, a powder, and a decontaminant.

12. A method of laundering soiled fabrics comprising the step of contacting said soiled fabrics with an effective amount of the composition of claim 8 or 9 in an aqueous solution.

13. The composition of claim 8 or 9, wherein the personal care component is selected from the group consisting of an oil, an emollient, a moisturizer, a carrier, an extract, a vitamin, a mineral, an anti-aging compound, a surfactant, a solvent, a polymer, a preservative, an antimicrobial, a wax, a particle, a colorant, a dye, a fragrance, and mixtures thereof.

14. The composition of claim 8 or 9, wherein the composition is selected from the group consisting of a shampoo, a hair conditioner, a hair treatment, a facial soap, a body wash, a body soap, a foam bath, a make-up remover, a skin care product, an acne control product, a deodorant, an antiperspirant, a shaving aid, a cosmetic, a depilatory, a fragrance, a lotion, and a mixtures thereof.

15. A method of treating, cleansing, or conditioning skin or hair comprising contacting the skin or hair with an effective amount of the composition of claim 8 or 9.
